(19) United States
(12) Patent Application Publication
(10) Pub. No.: US 2013/0150909 A1
(43) Pub. Date: Jun. 13, 2013

(54) MODES OF OPERATION FOR ATRIAL DEFIBRILLATION USING AN IMPLANTABLE DEFIBRILLATOR

(71) Applicants: Lazaro Salomon Azar, Givatayim (IL); Avi Allon Livnat, Tel Aviv (IL)

(72) Inventors: Lazaro Salomon Azar, Givatayim (IL); Avi Allon Livnat, Tel Aviv (IL)

(73) Assignee: SMARTWAVE MEDICAL LTD., Tel-Aviv (IL)

(21) Appl. No.: 13/636,587

(22) PCT Filed: Mar. 23, 2011
(86) PCT No.: PCT/US11/29509

§ 371 Date: Feb. 25, 2013

Related U.S. Application Data
(60) Provisional application No. 61/340,780, filed on Mar. 23, 2010.

(51) Int. Cl. A61N 1/39

(52) U.S. Cl. CPC A61N 1/395
USPC 607/7; 607/5

(57) ABSTRACT

Systems, methods and devices relating to atrial defibrillation and, more specifically, modes of operation for automatically and/or remotely causing the delivery of one or more atrial defibrillation pulses are disclosed. Embodiments provide for wireless communication between an implanted atrial defibrillator, external communication devices and/or servers to detect atrial fibrillation states, communicate with patients and/or initiate the delivery of atrial defibrillation pulses.

### CROSS-REFERENCE TO RELATED APPLICATIONS

The present application claims priority to U.S. Provisional Patent Application No. 61/340,780, filed on Mar. 23, 2010 and entitled “System and Method for Atrial Defibrillation Using an Implantable Defibrillator,” the disclosure of which is incorporated herein by reference in its entirety.

### FIELD

Systems, methods and devices for defibrillation are described herein. Some embodiments specifically relate to modes of operation for defibrillating the atria using an implantable defibrillator. Some embodiments more specifically relate to remote modes of operation enabling wireless communication between an implantable atrial defibrillator and a server.

### BACKGROUND

Atrial fibrillation (“AF”) is the most common cardiac arrhythmia involving at least one of the right atrium or left atrium. One way to defibrillate an atrium is by delivering electrical defibrillation pulses to the heart at specific times during the cardiac cycle. Systems and devices for delivering these pulses may be external and/or implanted within the body. Atrial defibrillation using an implantable atrial defibrillator generally includes automatically detecting AF and automatically delivering an electrical pulse to the left and/or right atrium. Delivering an electrical pulse however may be intolerably painful for a patient and, thus, may discourage the use of automatic implantable atrial defibrillators. While delivering an electrical pulse having an energy that is too high may cause pain to a patient, delivering an electrical pulse having an energy that is too low will result in an unsuccessful defibrillation attempt. Accordingly, atrial defibrillation that is tolerable and effective, and/or reduces the discomfort to a patient is desired.

### SUMMARY

In some embodiments described herein, a defibrillation system may include an implantable atrial defibrillator configured to monitor cardiac activity of the heart of a patient and collect atrial fibrillation data, an external communication device in wireless communication with the implantable atrial defibrillator for receiving the atrial fibrillation data and a server in wireless communication with the external communication device. The server may be configured to receive and analyze atrial fibrillation data and also determine whether the heart of the patient is in an atrial fibrillating state. In some embodiments, the server may communicate with the implantable atrial defibrillator in response to receiving and analyzing the atrial fibrillation data. The server may also cause the implantable atrial defibrillator to deliver at least one defibrillation pulse when the heart of the patient is in an atrial fibrillating state. In some embodiments, the at least one defibrillation pulse may be delivered to the heart at least one hour after the atrial defibrillating state was detected.

The implantable atrial defibrillator may be configured with a plurality of modes of operation that include at least one of a remote mode and an automatic mode. In some embodiments, the implantable atrial defibrillator may include both a remote mode and an automatic mode. When the implantable atrial defibrillator is set to the remote mode, the implantable atrial defibrillator may wirelessly transmit the atrial fibrillation data to the external communication device and/or wirelessly transmit the atrial fibrillation data to an interface device. In some embodiments, the interface device may be in wireless communication with at least one of the external communication device and the server. According to some embodiments, when the implantable atrial defibrillator is set to the remote mode, the implantable atrial defibrillator may wirelessly transmit the atrial fibrillation data directly to the server.

When the implantable atrial defibrillator is set to the automatic mode, the implantable atrial defibrillator may be configured to detect a state of atrial fibrillation and automatically deliver one or more atrial defibrillation pulses to the heart upon detection of atrial fibrillation. In some embodiments, the one or more atrial defibrillation pulses may be automatically delivered to the heart at least one hour after the detection of the atrial fibrillation.

In some embodiments, the implantable atrial defibrillator may notify the patient that atrial fibrillation has been detected. Notification means may include at least one of the implantable atrial defibrillator, the external communication device and the interface device.

In some embodiments, the external communication device may transmit and receive at least one of data and voice information. At least one of the external communication device and interface device may also have user inputs, including without limitation, a keypad, touch screen, scroll wheel and/or microphone, and/or user outputs, including without limitation, a display screen, speaker, vibrating mechanism and/or light-emitting component. The server may include a medical facility with computer equipment or human personnel that receive and analyze the atrial fibrillation data and, in some embodiments, may deploy personnel to visit the patient.

Some embodiments of the present disclosure may be directed to defibrillation methods. Methods may include monitoring the cardiac activity of the heart of a patient using an implantable atrial defibrillator, collecting data relating to the cardiac activity, detecting that the heart of the patient may be in a state of atrial fibrillation and determining whether the implantable atrial defibrillator has been set to at least one of a remote mode and automatic mode. In some embodiments, the remote mode may cause the implantable atrial defibrillator to be in wireless communication with at least one of an external communication device and a server and the automatic mode may cause the implantable atrial defibrillator to automatically deliver one or more atrial defibrillation pulses to the heart when a state of atrial fibrillation is detected. Some method embodiments may involve delivering the one or more atrial defibrillation pulses to the heart automatically at least one hour after a state of atrial fibrillation has been detected.

When the implantable atrial defibrillator is set to the remote mode, the implantable atrial defibrillator may wirelessly transmit the data relating to the cardiac activity to the external communication device and/or wirelessly transmit the data relating to the cardiac activity to the server. In some embodiments, the external communication device may wirelessly transmit the data relating to the cardiac activity to the server. When the implantable atrial defibrillator is set to the remote mode, the implantable atrial defibrillator may wirelessly transmit the atrial fibrillation data to an interface device. In some embodiments, the interface device may be in wireless communication with at least one of the external communication device and the server.

In some method embodiments, the implantable atrial defibrillator may notify the patient that atrial fibrillation has been detected. Notification means may include at least one of the implantable atrial defibrillator, the external communication device and the interface device.

In some embodiments, the server may include a medical facility with computer equipment or human personnel that analyze data relating to cardiac activity received from at least one of the implantable atrial defibrillator and external communication device. Upon analyzing the data relating to cardiac activity received from at least one of the implantable atrial defibrillator and external communication device, the server may cause the implantable atrial defibrillator to deliver at least one defibrillation pulse to the heart of the patient. Some embodiments may involve the server communicating with the implantable atrial defibrillator after receiving the data relating to cardiac activity from at least one of the implantable atrial defibrillator and external communication device. These communications may include instructions for the patient to initiate the delivery of an atrial defibrillation pulse.

In some embodiments, the implantable atrial defibrillator may be configured to detect abnormal ventricular activity and automatically delivers one or more ventricular defibrillation pulses to the heart.

The present disclosure also contemplates atrial defibrillation devices that include a housing implanted in or near the heart of a patient, one or more electrodes in wired communication with the housing for detecting cardiac activity of the heart and a communication transceiver within or about the housing and configured to wirelessly communicate with one or more external devices. In some embodiments, the atrial defibrillation device may operate in a remote mode to cause the communication transceiver to wirelessly transmit data relating to cardiac activity of the heart to the one or more external devices and/or an automatic mode to cause the automatic delivery of one or more atrial defibrillation pulses to the heart when a state of atrial fibrillation is detected.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1a shows a block diagram of an implantable atrial defibrillator according to some embodiments of the present disclosure.

FIG. 1 b shows a block diagram of an implantable atrial defibrillator according to some embodiments of the present disclosure.

FIG. 2 shows a defibrillation system according to some embodiments of the present disclosure.

FIG. 3 shows a flow diagram of a method of atrial defibrillation using an implantable atrial defibrillator and a defibrillation system according to some embodiments of the present disclosure.

FIG. 4 shows a flow diagram of a method of defibrillation using an implantable atrial defibrillator and a defibrillation system according to some embodiments of the present disclosure.

Like reference symbols in the various drawings indicate like elements.

### DETAILED DESCRIPTION

The subject matter described herein is not limited in its application to the details set forth in the following disclosure or exemplified by the illustrative embodiments. The subject matter is capable of other embodiments and of being practiced or carried out in various ways. Moreover, features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination or as suitable in any other described embodiment of the present disclosure. Certain features described in the context of various embodiments are not to be considered essential features of those embodiments, unless the embodiment is inoperative without those elements.

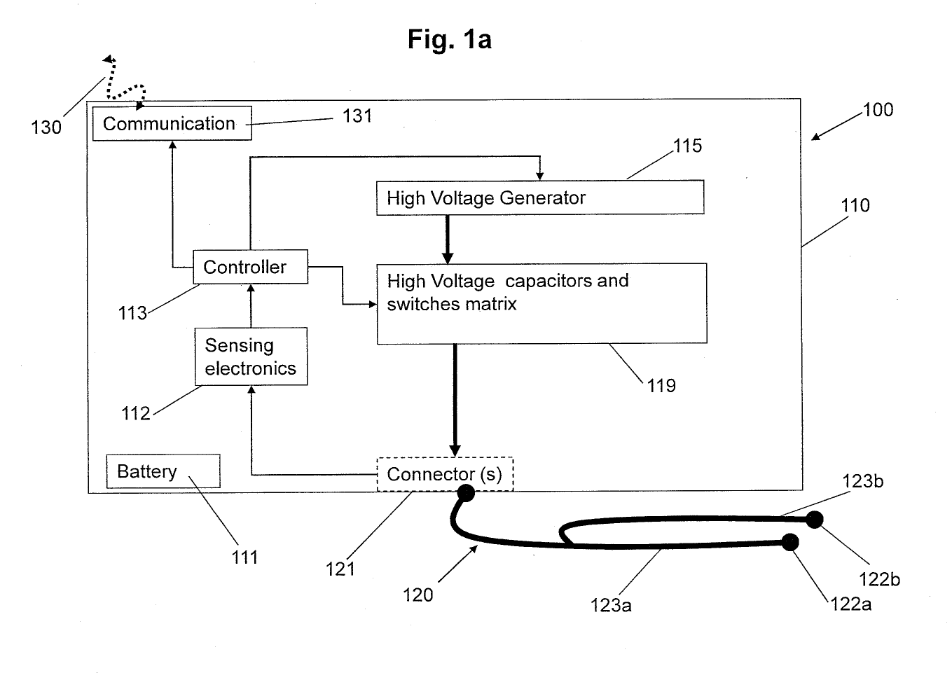

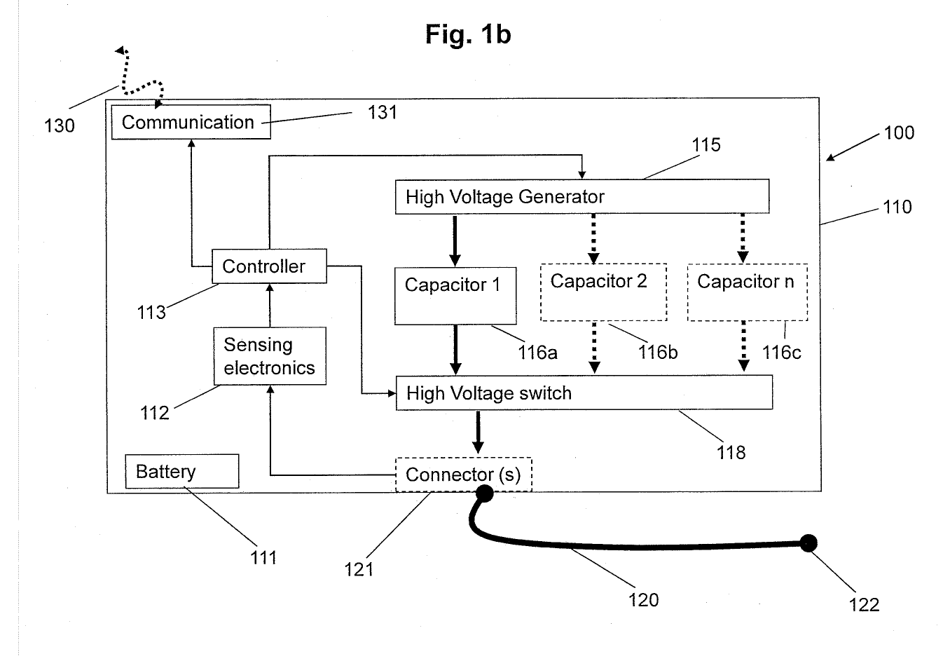

FIG. 1a shows a block diagram of an implantable atrial defibrillator (“IAD”) (100) according to some embodiments of the present disclosure. The internal construction of the IAD (100) may vary depending upon the embodiment and, in some embodiments, may be an internal construction that is known in the art. Example configurations of the IAD (100) are provided in International Publication No. WO2009/108502 to Livnat et al., filed on Feb. 11, 2009 and entitled “Atrial Defibrillation Using an Implantable Defibrillation System,” the disclosure of which is incorporated herein by reference in its entirety.

For performing the defibrillation methods contemplated by the present disclosure, the IAD (100) may include a communication transceiver (131) capable of wirelessly communicating with an external device using a communication link (130). The communication link (130) may have short-range and/or long-range capabilities. The communication link (130) may be an ultrasonic link communicating with an external device in contact with a patient's body. In some embodiments, the communication link (130) may be a short-range radio frequency (“RF”) communication link and may use a proprietary protocol for communicating with an interface device. In some embodiments, the communication link (130) may use a common protocol, such as Bluetooth technology or wireless fidelity (“Wi-Fi”), wherein the external device may include mobile devices (i.e., portable devices), such as, for example, a mobile phone, media player, smart phone, Personal Digital Assistant (“PDA”) and other handheld computing devices and the like.

The IAD (100) may have a main body (110). The main body (110) may be made of one or more bio-compatible materials known in the art. The main body (110) may contain at least one battery (111) and electronic circuitry for sensing cardiac activity, processing the sensed activity to determine whether the activity is normal or indicative of a fibrillation state, and delivering one or more high-voltage defibrillation pulses. In some embodiments, the IAD (100), and in particular the electronic circuitry may be configured to differentiate between atrial and ventricular fibrillations and respond accordingly based on whether the atria or ventricles of the heart are fibrillating.

Some embodiments of the main body (110) may include at least one electrical connector (121) connected to a lead (120). In some embodiments, the lead (120) may be permanently attached to the main body (110). In some embodiments, the lead (120) may be bifurcated into sub-leads 123a and 123b having exposed electrodes (122a) and (122b), respectively. The number of leads (120), sub-leads (123) and electrodes (122), as well as their specific configurations, may vary depending on the embodiment. The locations of the electrodes (122) along the leads (120) and/or sub-leads (123) may also vary depending on the embodiment. For example, some embodiments of the IAD (100) may position one or more electrodes in left and/or right atrium for pacing the heart, in addition to those electrodes used for atrial defibrillation. In some embodiments, one or more additional electrodes may be positioned in the right ventricle may be used for electrocardiogram (“ECG”) sensing and delivering one or more ventricular defibrillation pulses or pulse trains. In some embodiments, the main body (110), or parts thereof, may be used as an electrode. In some embodiments, the communication transceiver (131) may use the lead (120) as an antenna for RF communication. Some embodiments of the IAD (100) may include a dedicated antenna, for example a coil, loop or dipole antenna, located within or outside the main body (110).

At least one of the electrodes (122) may be used for sensing ECG signals for monitoring the cardiac activity of a patient implanted with the IAD (100). In some embodiments, one or more of the same electrodes (122) may be used for both sensing ECG data and delivering defibrillation pulses or cardiac pacing. In some embodiments, at least one electrode (122) may be dedicated to sensing ECG signals. Embodiments of the IAD (100) may include a sensing electronic module (112) configured to condition (e.g., amplify and/or filter) the ECG signals. The IAD (100) may include additional sensors for monitoring cardiac activity and other bodily functions. For example, the IAD (100) may include one or more thermal sensors to monitor patient body temperature, blood oxygenation sensors, microphones to monitor sound emitted from the heart and the respiratory system, breathing sensors (e.g., capacitive sensors or sensors sensing the bending of the lead (120) due to breathing) and/or other sensors known in the art. In some embodiments, sensor electronics may include an Analog-to-Digital Converter (“ADC”).

The IAD (100) may include a controller (113) for performing signal conditioning and analysis. The controller (113) may receive data indicative of cardiac activity from the sensing electronics (112) and other optional sensors and may receive commands and data from the communication transceiver (131). The controller (113) may determine the state of the cardiac activity based on ECG signals and other sensor data and control the pulse-generating circuitry to produce one or more defibrillation pulses when appropriate. In some embodiments, pulse-generating circuitry may include a high-voltage generator (115) and a high-voltage capacitor and switches matrix (119) configured to produce high-voltage, short-duration pulses for defibrillating the atria and/or ventricles of the heart. In some embodiments, atrial defibrillation may be done using low-energy (e.g., <2 J), high-voltage (e.g., >600 V), short-duration (e.g., <100 μs) pulses. Other exemplary energy, voltage and/or pulse duration ranges are set forth in co-pending U.S. Provisional Patent Application No. 61/416,964, filed Nov. 24, 2010 and entitled “Implantable Defibrillation System,” the content of which is hereby incorporated by reference in its entirety. In other embodiments, a train of two or more pulses may be used. In some embodiments, the IAD (100) may be configured as an atrial defibrillator and pacemaker, an atrial defibrillator and ventricular defibrillator (also known as an implantable cardioverter-defibrillator, or “ICD”) or an atrial defibrillator, ventricular defibrillator and pacemaker. The IAD (100) may be able to monitor, detect and collect data relating to cardiac activity, analyze whether a cardiac condition exists and deliver a defibrillation and/or pacing therapy that best treats the condition. Analyzing the cardiac activity and identifying the existence of a condition may be performed by the controller (113) of the IAD (100), in conjunction with other circuitry and software within the IAD (100). Alternatively, or in addition, cardiac activity analyses and processing may be performed remotely by a medical facility that receives the collected data over the communication link (130).

FIG. 1b shows a block diagram of an embodiment of the IAD (100) of FIG. 1a according to the present disclosure. In this embodiment, high-voltage capacitor and switches matrix (119) may include at least one high-voltage capacitor (116a) capable of being charged to a desired high voltage by high-voltage generator (115). The high-voltage capacitor and switches matrix (119) may also include a high-voltage switch (118) that discharges voltage stored in the high-voltage capacitor (116a) into the lead (120). The high-voltage switch (118) may control pulse duration. The high-voltage and switches matrix (119) may include additional high-voltage capacitors (116b) and (116c) for generating a train of pulses. The pulses in the train may have the same or opposite polarity and/or different voltage and duration. Some embodiments of the IAD (100) may have a patient notification element (133), such as a vibrator or buzzer, to alert a patient when AF has been detected.

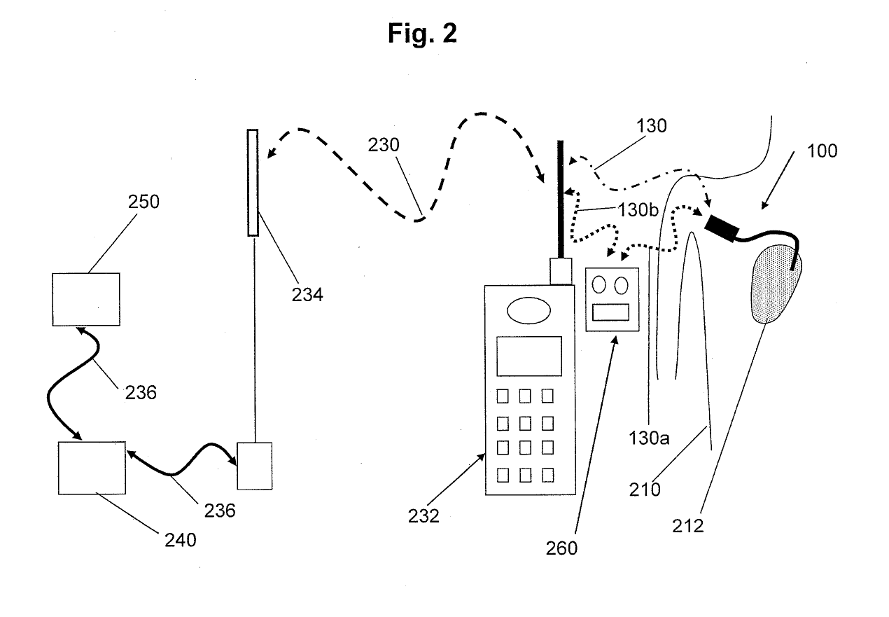

FIG. 2 shows a defibrillation system (200) using an embodiment of the IAD (100) according to the subject matter of the present disclosure. In some embodiments of the system (200), the IAD (100) may be implanted in a patient (210). One or more electrodes (122) may be positioned in or around the atria of the heart (212) of the patient (210). The system (200) also includes an external communication device (232), an interface device (260) and a server (240), all of which may be in wireless communication with one another. In some embodiments, the IAD (100) may communicate directly with the server (240) or via the external communication device (232) and/or the interface device (260) to, for example, transmit data to the server (240) relating to a possible AF state.

The IAD (100) of the system (200) may communicate with the external communication device (232). The communication between the IAD (100) and the external communication device (232) may be short-range and/or long-range communication. The external communication device (232) may be configured as a two-way communicator capable of transmitting and receiving both data and voice information or, alternatively, the external communication device (232) may be configured to transmit and receive only data or only voice information. In some embodiments, the external communication device (232) may include one or more user inputs, such as a keypad, touch screen, scroll wheel or microphone. Some embodiments of the external communication device (232) may have one or more user outputs, such as a display screen, speaker, vibrating mechanism and/or light-emitting component (e.g., a light-emitting diode). The external communication device (232) may also include a global positioning system (“GPS”) receiver for determining the location of the external communication device (232). The external communication device (232) may be a cellular phone, a smartphone or any other handheld computing device. In some embodiments, external communication device (232) may also be a satellite communication device.

In some embodiments, the IAD (100) may communicate with the external communication device (232) via the communication link (130), as shown in FIG. 2. The IAD (100) may, in some embodiments, communicate with the external communication device (232) via an interface device (260). In some embodiments, the interface device (260) may be an application embedded within external communication device (232). In some embodiments, the external communication device (232) and/or the interface device (260) may be embedded within the IAD (100) itself, either as software and/or hardware components of the IAD (100). Other embodiments of the present disclosure contemplate the interface device (260) as a separate component in wireless communication with IAD (100), server (240) and/or external communication device (232). In such embodiments, the interface device (260) may be any shape or size. The interface device (260) may be miniature for discreet placement in or around the heart (212) of the patient (210). The interface device (260), in some embodiments, may be used primarily for providing an interface between the IAD (100) and the external communication device (232) and, thus, may contain no user inputs or outputs. In other embodiments, the interface device (260) may communicate directly with the server (240). The interface device (260) may include user inputs, such as switches or buttons, and user outputs, such as a display screen, speaker(s) and/or vibrating mechanism. Communication between the IAD (100) and the external communication device (232) via the interface device (260) may involve using short-range channels. As shown in FIG. 2, the IAD (100) may communicate with the interface device (260) via a short-range channel (130a) and the interface device (260) may communicate with the external communication device (232) via a short-range channel (130b). In some embodiments, the channels connecting the IAD (100), interface device (260) and external communication device (232) may be long-range channels or a combination of short-range and long-range channels.

FIG. 2 also shows that the external communication device (232) may communicate with the server (240) via a long-range communication channel (230). For example, the external communication device (232) may be a mobile phone that communicates with a base station (234) over a long-range communication channel (230), such as a cellular RF channel, and connect to the server (240) over a channel (236). The channel (236) may be a land line, cellular line or other communication channel, such as the Internet. In some embodiments, the external communication device (232) may be a satellite communication device capable of communicating with the server (240) from anywhere around the world. The server (240) may constitute a medical center, hospital and the like, as well as any computers, hospital equipment and human personnel located at any such facility.

In some embodiments, the server (240) may communicate with a rescue team (250) (e.g., a medical team, paramedics and/or an ambulance) over the channel (236) (e.g., land or cellular lines) and direct the rescue team (250) to the location of the patient (210). In some embodiments, the external communication device (232) may communicate directly with the rescue team (250).

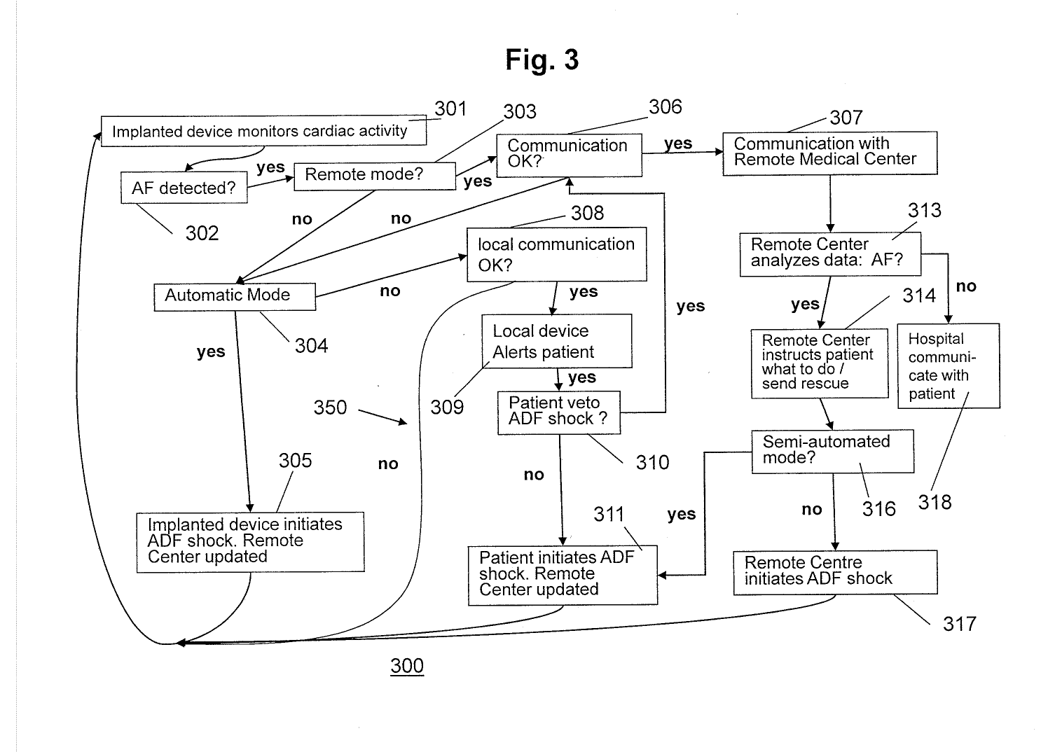

FIG. 3 shows a flow diagram of a method (300) of defibrillating the atria using embodiments of the system (200) and the IAD (100) in accordance with the subject matter of the present disclosure. The method (300) begins at a monitoring step (301), where the IAD (100) monitors the cardiac activity of the heart of the patient (210) in FIG. 2. If an AF state is detected (or suspected) by the IAD (100) (see step 302), the IAD (100) may respond in various ways depending on how the IAD (100) is configured. In some embodiments, the IAD (100) may be configured with a remote mode (303) and/or an automatic mode (304). When the IAD (100) is set in automatic mode (304), the IAD (100) will not attempt to communicate with the interface device (260) and/or the external communication device (232). Rather, in the automatic mode (304), the IAD (100) will automatically deliver an atrial defibrillation pulse to the heart, as shown at delivery step (305) in FIG. 3. In some embodiments, the method (300) may be configured to determine a clinically-optimal time for defibrillating the atria. For example, some embodiments may deliver a defibrillation pulse immediately at or after the onset and/or detection of an AF state. In other embodiments, a defibrillation pulse may be delivered at some length of time (e.g., 1 hour, 4 hours or 16 hours) after the onset and/or detection of an AF state. After delivering a defibrillation pulse to the atria, the IAD (100) may resume monitoring cardiac activity at the monitoring step (301). In some embodiments, the IAD (100) may store information pertaining to the defibrillation episode, including without limitation, ECG data from before and/or after the defibrillation and the time, date and parameters of the defibrillation. This information, along with other information, may be relayed to the server (240) when communication is established between the server (240) and the IAD (100) (e.g., directly or via external communication device (232) and/or interface device (260)).

When the IAD (100) is in remote mode (303), the IAD (100) will attempt to communicate (see step 306) with the server (240) to transmit information regarding the AF state to the server (240) (e.g., a remote medical center) (see step 307). The IAD (100) may attempt to communicate directly with the server (240) or by way of the external communication device (232) and/or the interface device (260) using communication link (230), base station (234) and/or channel (236). If the IAD (100) establishes communication with the server (240), the server (240) may receive AF data from the IAD (100). The AF data may contain, for example, information about the AF state of the patient (210) as determined by the sensing electronics (112) (see FIGS. 1a, 1b), measured ECG data, data measured by other sensors in the IAD (100) (e.g., temperature and/or acceleration) and/or the state of the IAD (100) (e.g., battery charge status and/or planed pulse parameters). In some embodiments, the IAD (100) may transmit unprocessed and/or minimally processed information, such as raw ECG data and/or other sensor readings. In some embodiments, the data transmitted by the IAD (100) may be encoded, encrypted and/or compressed.

The server (240), and/or human personnel located at the server (240), may process and analyze the received AF data (see step 313) to determine one or more appropriate courses of action. In some method embodiments, the server (240) may perform this analysis automatically. In other embodiments, human data analysis and/or decision-making may be required or preferred. For example, a cardiologist may interpret transmitted ECG data to determine the cardiac state of the patient (210). The server (240) may obtain or store health records of the patient (210), such as general health data and ECG records from previous AF episodes and normal ECG records, to assist in determining the best course of action. The location of the patient (210) may be determined based on cellular network technology and/or a GPS receiver. Based on this information, the server (240) or individuals at the server (240) may determine whether the patient (210) has time to get to a medical facility or if a rescue team must be deployed. In some embodiments, when it is determined that an AF state exists, the server (240) may communicate (see step 314) with the patient (210). For example, the patient (210) may be consulted and asked about his/her health condition, general situation and preferred course of action. The patient may be requested to perform tasks, such as rest, take medication (e.g., to correct the AF state or prepare for defibrillation pulses), perform a medical examination (e.g., obtain ECG data or transmit pulse information by placing the microphone of the interface device (260) or the external communication device (232) on his/her chest), wait for a rescue team (250), stop driving, or go to the nearest or a selected medical facility. In some method embodiments, a rescue team (250) may be sent to the location of the patient (210). The rescue team (250) may be informed of the status of the patient (210) and receive continual status updates prior to arrival.

When it has been determined that an AF state exists, the server (240) may communicate with the IAD (100) and cause the IAD (100) to deliver a defibrillation pulse to the patient (210) (see step 317). In some embodiments, the IAD (100) may be in a semi-automatic mode (316), wherein the patient may be asked or instructed by the server (240) to initiate the delivery of a defibrillation pulse using the interface device (260) or the external communication device (232). The server (240) may determine a clinically-optimal time for delivering defibrillation pulses and initiate the delivery of one or more pulses at such optimal time either automatically or via instructions to the patient (210). For example, some embodiments may initiate the delivery of a defibrillation pulse immediately at or after the onset and/or detection of an AF state. In other embodiments, a defibrillation pulse may be delivered at some length of time (e.g., 1 hour, 4 hours or 16 hours) after the onset and/or detection of an AF state. The IAD (100) may store information pertaining to the defibrillation episode, including without limitation, ECG data from before and/or after the defibrillation and the time, date and parameters of the defibrillation. This information, along with other information, may be relayed to the server (240) when communication is established between the server (240) and the IAD (100) (e.g., directly or via external communication device (232) and/or interface device (260)).

If the server (240) determines that an AF state does not exist, the server (240) may communicate with the patient (210) (see step 318) to reassure the patient (210) that his/her cardiac conditions are normal. This ability to communicate with the patient (210) is advantageous when the patient (210) initiates communication with the server (240), for example, as a routine system check or due to pain or other symptoms. The server (240) may communicate with the patient (210) and/or instruct people near the patient (210) (e.g., using high-volume speakers on the external communication device (232)) to assist the patient (210) accordingly.

When the IAD (100) is in neither remote mode (303), nor automatic mode (304), the IAD (100) may try to communicate locally with the patient (210) to alert the patient (210) that atrial defibrillation has been detected. At local communication step (308), the IAD (100) may determine whether it is configured to communicate locally with the patient (210). For example, the IAD (100) may determine whether it has the ability to alert the patient (210) about an AF state using a local mechanism on the IAD (100), such as the patient notification element (133) (see FIG. 1b), or by an external mechanism on the external communication device (232) and/or the interface device (260). If local communication (308) with the patient (210) is not possible or fails (e.g., the patient (210) does not hear/notice the alert signals) and the remote mode (303) and the automatic mode (304) are turned off, the IAD (100) may return to the monitoring step (301) to monitor the cardiac activity without delivering an atrial defibrillation pulse, as shown by reference number 350.

When local communication (308) is possible and successfully alerts the patient (210) that an AF state may exist (see step 309), the patient (210) may have the choice (see step 310) to confirm and initiate the delivery of a defibrillation pulse or veto such delivery. If the patient (210) confirms and initiates delivery, a defibrillation pulse will be delivered to the heart (212). In some embodiments, the method (300) may be configured to determine a clinically-optimal time for defibrillating the atria. For example, some embodiments may deliver a defibrillation pulse immediately at or after the onset and/or detection of an AF state. In other embodiments, a defibrillation pulse may be delivered at some length of time (e.g., 1 hour, 4 hours or 16 hours) after the onset and/or detection of an AF state. Thereafter, the IAD (100) may continue monitoring the heart (212) at monitoring step (301). The IAD (100) may store information pertaining to the defibrillation episode, including without limitation, ECG data from before and/or after the defibrillation and the time, date and parameters of the defibrillation. This information, along with other information, may be relayed to the server (240) when communication is established between the server (240) and the IAD (100) (e.g., directly or via external communication device (232) and/or interface device (260)).

If the patient (210) vetoes the delivery of a defibrillation pulse, the IAD (100) may automatically switch to remote mode (303) to attempt to communicate (see step 306) with the server (240) to transmit information regarding the AF state to the server (240) (see step 307), wherein the remote mode (303) protocols described above are possible. In some situations, depending on the detected severity of the cardiac state, the IAD (100) may automatically switch to automatic mode (304) to delivery defibrillation pulses immediately. In some embodiments, the defibrillation pulses may be delivered immediately at or after the onset and/or detection of an AF state. In other embodiments, the pulses may be delivered at some length of time (e.g., 1 hour, 4 hours or 16 hours) after the onset and/or detection of an AF state. Similar protocols may apply when the patient (210) may choose to do nothing, i.e., neither confirms nor vetoes the delivery of a defibrillation pulse. Because AF typically poses little, if any, life-threatening danger to a patient, it may be advantageous not to automatically deliver a defibrillation pulse to an unprepared patient to avoid startling the patient and/or causing an accident. Rather, monitoring (step 301) should continue or, at most, the IAD (100) should go into remote mode (303). However, if the IAD (100) detects a life-threatening cardiac state, such as prolonged and/or strong AF or abnormal ventricular activity, the IAD (100) may switch into automatic mode (304) (even if local communication with the patient (210) is not available) to commence corrective steps, such as heart pacing, atrial defibrillation or ventricular defibrillation.

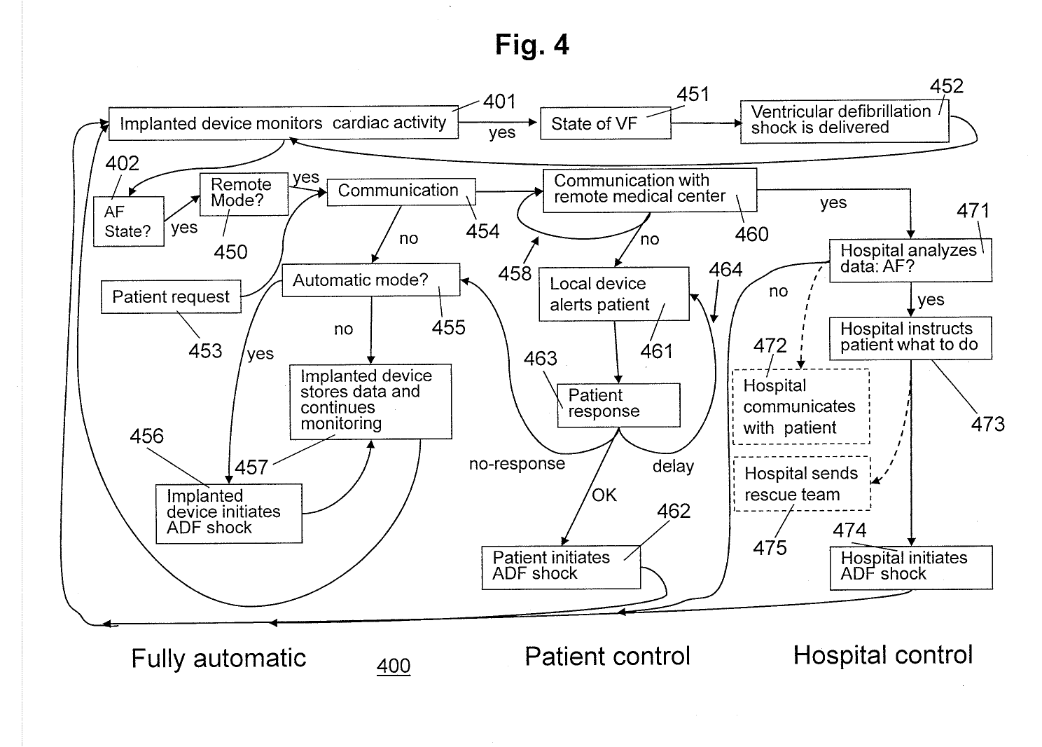

FIG. 4 shows a flow diagram of a method (400) of defibrillation using embodiments of the defibrillation system (200) and the IAD (100) in accordance with the subject matter of the present disclosure. The IAD (100) may monitor the cardiac activity of the patient (210) at monitoring step (401) to detect states of AF. In some embodiments, the IAD (100) may be configured to also detect and treat ventricular fibrillation (“VF”), as shown at reference numeral 451. If VF is detected, a ventricular defibrillation pulse may be delivered at delivery step (452). In some embodiments, the IAD (100) may respond to a state of VF as any implanted ventricular defibrillator known in the art would respond.

When AF is detected by the IAD (100) at step 402, and the IAD (100) is set to a remote mode (450), the IAD (100) may attempt to communicate (see step 454) with the external communication device (232) and/or the interface device (260). If such communication (454) is established, the IAD (100) may transmit a message containing, for example, information about the AF state of the patient (210) as determined by the sensing electronics (112) (see FIGS. 1a, 1b), measured ECG data, data measured by other sensors in the IAD (100) (e.g., temperature and/or acceleration) and/or the state of the IAD (100) (e.g., battery charge status and/or planed pulse parameters). In some embodiments, the IAD (100) may transmit unprocessed and/or minimally processed information, such as raw ECG data and/or other sensor readings. In some embodiments, the data transmitted by the IAD (100) may be encoded, encrypted and/or compressed.

Data transmission may be initiated by a command sent to the IAD (100), for example, using the external communication device (232) and/or the interface device (260). In some method embodiments, the patient (210), or a person near him, may use the external communication device (232) and/or the interface device (260) to request or initiate (see step 453) data transmission from the IAD (100) to the external communication device (232) and/or interface device (260). For example, the patient (210) may use this option as a routine check of the IAD (100) and/or to update the server (240) with current ECG data. The patient (210) may request or initiate (see step 453) data transmission from the IAD (100) directly to the server (240) or to the server (240) via the external communication device (232) and/or interface device (260) when he/she feels sick or has symptoms that concern him/her. The server (240) itself may also request or initiate (see step 453) data transmission from the IAD (100) to the external communication device (232) and/or interface device (260), for example, as a routine check or if the patient (210) informed the server (240) of his/her concerns.

When an AF state has been detected at step 402, but attempts by the IAD (100) to communicate (see step 454) with the external communication device (232) and/or the interface device (260) have failed, the IAD (100) may check an internal setting to determine if it is configured to operate in an automatic mode (455), as shown in FIG. 4. In automatic mode (455), the IAD (100) may deliver an atrial defibrillation pulse to the patient (210) (see step 457). In some embodiments, the IAD (100) may be configured to determine a clinically-optimal time for delivering the pulse. Some embodiments may deliver the pulse immediately at or after the onset and/or detection of an AF state. In other embodiments, a pulse may be delivered at some length of time (e.g., 1 hour, 4 hours or 16 hours) after the onset and/or detection of an AF state. In some embodiments, the IAD (100) may confirm the state of AF before delivering an atrial defibrillation pulse. The IAD (100) may store information (see step 457) pertaining to the AF episode, attempts to communicate with the server (240) and any defibrillation episodes. Following delivery of an atrial defibrillation pulse, the IAD (100) may continue to monitor the cardiac activity at monitoring step (401). Even if the IAD (100) is not configured with and/or set to automatic mode (455), it may still store information (see step 457) pertaining to the AF episode and the attempts to communicate with the server (240). The automatic mode (455) may be configured by the manufacturer at the factory and/or by a user (e.g., physician) before implantation. The automatic mode (455) may also be changed after implantation.

If the IAD (100) establishes communication (see step 454) with the external communication device (232) and/or the interface device (260), the external communication device (232) may attempt to communicate with the server (240), as shown at step 460 in FIG. 4. If the attempt fails, the external communication device (232) may continue to attempt communicate with the server (240), as shown at reference numeral 458 in FIG. 4. In the interim, or alternatively, the external communication device (232) and/or the interface device (260) may alert the patient (210) at step 461 of an AF state using, for example, vibration, light and/or sound. Following such an alert, the IAD (100) may wait for the patient (210) to respond. The patient (210) may respond by confirming and initiating the delivery of an atrial defibrillation pulse, wherein a pulse is delivered (see step 462) and monitoring (step 401) is thereafter continued. In some embodiments, the pulse may be delivered immediately at or after the onset and/or detection of an AF state. In other embodiments, the pulse may be delivered at some length of time (e.g., 1 hour, 4 hours or 16 hours) after the onset and/or detection of an AF state. The IAD (100) may store information pertaining to the defibrillation episode, including without limitation, ECG data from before and/or after the defibrillation and the time, date and parameters of the defibrillation. This information, along with other information, may be relayed to the server (240) when communication is established between the server (240) and the IAD (100).

In some embodiments, the patient (210) may respond with a command to delay the delivery of an atrial defibrillation pulse, as shown at step 464. Following such a command, the IAD (100) may continue to monitor the cardiac activity of the patient (210), as well as wait to receive a command from the patient (210) to initiate delivery of an atrial defibrillation pulse. In some embodiments, if no patient response is received within a predetermined time, or if communication between the IAD (100) and the external communication device (232) and/or the interface device (260) is disconnected, the IAD (100) may switch to the automatic mode (455) and act as “stand-alone device.” In some embodiments, a different preset time may be assigned for receiving an additional patient response after already receiving an initial command from the patient (210) to delay pulse delivery.

If the external communication device (232) establishes communication with the server (240) (see step 460), the server (240) may analyze the data sent from the IAD (100), as shown at step 471, to determine if an AF state exists. The server (240), in some embodiments, may refer the case to human personnel for further examination. If the server (240) determines that a patient's health was not compromised by the AF state, it may reset the IAD (100) to the monitoring state (step 401). The server (240) may communicate with the patient (210) to reassure him/her, request more information and/or give instructions, as shown at step 472 in FIG. 4.

Alternatively, if an AF state is detected by the server (240), the server (240) may communicate with the patient (210) to, for example, gather information about the health condition of the patient (210), the general situation and/or any preferred course of action. The patient (210) may be requested to perform tasks, such as rest, take medication (e.g., to correct AF state or prepare for defibrillation pulses), perform a medical examination (e.g., obtain ECG data or transmit pulse information by placing the microphone of the interface device (260) or the external communication device (232) on his/her chest), wait for a rescue team, stop driving or go to the nearest or a selected medical facility. In some method embodiments, the rescue team (250) may be sent to the location of the patient (210), as shown at step 475. The rescue team (250) may be informed of the status of the patient (210) and receive continual status updates prior to arrival.

In some embodiments, when it has been determined by the server (240) that an AF state exists (see step 471), the server (240) may communicate directly with the IAD (100) or via the external communication device (232) and/or interface device (260) to cause the IAD (100) to deliver a defibrillation pulse to the patient (210). In some embodiments, the IAD (100) may be in a semi-automatic mode, wherein the patient may be asked or instructed by the server (240) to initiate the delivery of a defibrillation pulse using the interface device (260) and/or the external communication device (232). The server (240) may determine a clinically-optimal time for delivering defibrillation pulses and initiate the delivery of one or more pulses at such optimal time either automatically or via instructions to the patient (210). For example, some embodiments may initiate the delivery of a defibrillation pulse immediately at or after the onset and/or detection of an AF state. In other embodiments, a defibrillation pulse may be delivered at some length of time (e.g., 1 hour, 4 hours or 16 hours) after the onset and/or detection of an AF state. The IAD (100) may store information pertaining to the defibrillation episode, including without limitation, ECG data from before and/or after the defibrillation and the time, date and parameters of the defibrillation. This information, along with other information, may be relayed to the server (240) when communication is established between the server (240) and IAD (100).

It should be noted that the steps and order of steps described with respect to FIGS. 3 and 4 may be combined in various ways within the general scope of the present disclosure. Additional steps may be added and some may be changed or missing.

The embodiments set forth in the foregoing description do not represent all embodiments consistent with the subject matter described herein. It is evident that many alternatives, modifications and variations of such embodiments will be apparent to those skilled in the art. As noted elsewhere, these embodiments have been described for illustrative purposes only and are not intended to be limiting. Thus, other embodiments are possible and are covered by the disclosure, which will be apparent from the teachings contained herein. The breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices; that is, elements from one or another of the disclosed embodiments may be interchangeable with elements from another of the disclosed embodiments. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to any of the disclosed embodiments.

What is claimed is:

1. A defibrillation system comprising:
an implantable atrial defibrillator configured to monitor cardiac activity of the heart of a patient and collect atrial fibrillation data;
an external communication device in wireless communication with the implantable atrial defibrillator for receiving the atrial fibrillation data; and
a server in wireless communication with the external communication device, the server being configured to:
receive and analyze the atrial fibrillation data; and
determine whether the heart of the patient is in an atrial fibrillating state,
wherein the server communicates with the implantable atrial defibrillator in response to receiving and analyzing the atrial fibrillation data.

2. The defibrillation system of claim 1, wherein the server causes the implantable atrial defibrillator to deliver at least one defibrillation pulse when the heart of the patient is in an atrial fibrillating state.

3. The defibrillation system of claim 2, wherein the at least one defibrillation pulse is delivered to the heart of the patient at least one hour after the detection of the atrial fibrillating state.

4. The defibrillation system of claim 1, wherein the implantable atrial defibrillator is configured with a plurality of modes of operation that include at least one of a remote mode and an automatic mode.

5. The defibrillation system of claim 1, wherein the implantable atrial defibrillator is configured with a plurality of modes of operation that include a remote mode and an automatic mode

6. The defibrillation system of claim 4, wherein, when the implantable atrial defibrillator is set to the remote mode, the implantable atrial defibrillator wirelessly transmits the atrial fibrillation data to the external communication device.

7. The defibrillation system of claim 4, wherein, when the implantable atrial defibrillator is set to the remote mode, the implantable atrial defibrillator wirelessly transmits the atrial fibrillation data to an interface device.

8. The defibrillation system of claim 7, wherein the interface device is in wireless communication with at least one of the external communication device and the server.

9. The defibrillation system of claim 1, wherein, when the implantable atrial defibrillator is set to the remote mode, the implantable atrial defibrillator wirelessly transmits the atrial fibrillation data directly to the server.

10. The defibrillation system of claim 2, wherein, when the implantable atrial defibrillator is set to the automatic mode, the implantable atrial defibrillator is configured to detect a state of atrial fibrillation and automatically deliver one or more atrial defibrillation pulses to the heart upon detection of atrial fibrillation.

11. The defibrillation system of claim 10, wherein the one or more atrial defibrillation pulses are automatically delivered to the heart at least one hour after the detection of the atrial fibrillation.

12. The defibrillation system of claim 1, wherein the implantable atrial defibrillator notifies the patient that atrial fibrillation has been detected.

13. The defibrillation system of claim 12, wherein the patient is notified by a notification means on at least one of the implantable atrial defibrillator, the external communication device and an interface device.

14. The defibrillation system of claim 1, wherein the external communication device transmits and receives at least one of data and voice information.

15. The defibrillation system of claim 1, wherein at least one of the external communication device and an interface device includes user inputs selected from the group consisting of a keypad, touch screen, scroll wheel and microphone or user outputs selected from the group consisting of a display screen, speaker, vibrating mechanism and light-emitting component.

16. The defibrillation system of claim 1, wherein the server includes a medical facility with computer equipment or human personnel that receive and analyze the atrial fibrillation data.

17. The defibrillation system of claim 1, wherein the server deploys personnel to visit the patient.

18. A defibrillation method comprising:
monitoring the cardiac activity of the heart of a patient using an implantable atrial defibrillator;
collecting data relating to the cardiac activity;
detecting that the heart of the patient may be in a state of atrial fibrillation; and
determining whether the implantable atrial defibrillator has been set to at least one of a remote mode and automatic mode,
wherein:
the remote mode causes the implantable atrial defibrillator to be in wireless communication with at least one of an external communication device and a server; and
the automatic mode causes the implantable atrial defibrillator to automatically deliver one or more atrial defibrillation pulses to the heart when a state of atrial fibrillation is detected.

19. The method of claim 18, wherein, when the implantable atrial defibrillator is set to the remote mode, the implantable atrial defibrillator wirelessly transmits the data relating to the cardiac activity to the external communication device.

20. The method of claim 18, when the implantable atrial defibrillator is set to the remote mode, the implantable atrial defibrillator wirelessly transmits the data relating to the cardiac activity to the server.

21. The method of claim 19, wherein the external communication device wirelessly transmits the data relating to the cardiac activity to the server.

22. The method of claim 18, wherein, when the implantable atrial defibrillator is set to the remote mode, the implantable atrial defibrillator wirelessly transmits the atrial fibrillation data to an interface device.

23. The method of claim 22, wherein the interface device is in wireless communication with at least one of the external communication device and the server.

24. The method of claim 18, wherein the implantable atrial defibrillator notifies the patient that atrial fibrillation has been detected.

25. The method of claim 18, wherein the patient is notified by a notification means on at least one of the implantable atrial defibrillator, the external communication device and an interface device.

26. The method of claim 18, wherein at least one of the external communication device and an interface device includes user inputs selected from the group consisting of a keypad, touch screen, scroll wheel and microphone or user outputs selected from the group consisting of a display screen, speaker, vibrating mechanism and light-emitting component.

27. The method of claim 18, wherein the server includes a medical facility with computer equipment or human personnel that analyze data relating to cardiac activity received from at least one of the implantable atrial defibrillator and external communication device.

28. The method of claim 27, wherein, upon analyzing data relating to cardiac activity received from at least one of the implantable atrial defibrillator and external communication device, the server causes the implantable atrial defibrillator to deliver at least one defibrillation pulse to the heart of the patient.

29. The defibrillation system of claim 18, wherein the one or more atrial defibrillation pulses are delivered to the heart at least one hour after the state of atrial fibrillation has been detected.

30. The method of claim 28, wherein the server communicates with the implantable atrial defibrillator after receiving the data relating to cardiac activity from at least one of the implantable atrial defibrillator and external communication device.

31. The method of claim 30, wherein the communication from the server is an instruction for the patient to initiate the delivery of an atrial defibrillation pulse.

32. The method of claim 18, wherein the implantable atrial defibrillator detects abnormal ventricular activity and automatically delivers one or more ventricular defibrillation pulses to the heart.

33. An atrial defibrillation device comprising;
a housing implanted in or near the heart of a patient;
one or more electrodes in wired communication with the housing for detecting cardiac activity of the heart; and
a communication transceiver within or about the housing and configured to wirelessly communicate with one or more external devices,
wherein the atrial defibrillation device operates in at least one of:
a remote mode to cause the communication transceiver to wirelessly transmit data relating to cardiac activity of the heart to the one or more external devices; and
an automatic mode to cause the automatic delivery of one or more atrial defibrillation pulses to the heart when a state of atrial fibrillation is detected.
